(19) United States
(12) Patent Application Publication
(10) Pub. No.: US 2003/0221205 A1
(43) Pub. Date: Nov. 27, 2003

(54) Olfactory and pheromones G-protein coupled receptors

(76) Inventor: Alex Veithen, Nivelles (BE)

Correspondence Address: PALMER & DODGE, LLP, KATHLEEN M. WILLIAMS, 111 HUNTINGTON AVENUE, BOSTON, MA, 02199, US

(21) Appl. No.: 10/387,629

(22) Filed: Mar. 13, 2003

Related U.S. Application Data
(63) Continuation-in-part of application No. PCT/BE01/00162, filed on Sep. 21, 2001.

(30) Foreign Application Priority Data
Sep. 22, 2000 (EP) 008702311.0

(51) Int. Cl. A01K 67/027; C12Q 1/68; C07H 21/04; C07K 14/705; C12P 21/02; C12N 5/06; A61K 38/17

(52) U.S. Cl. 800/14; 435/6; 435/69.1; 435/320.1; 435/325; 530/350; 536/23.5; 514/12

(57) ABSTRACT

The present invention is related to an olfactory or pheromone G-protein coupled receptor having an amino acid sequence which presents 95% sequence identity with the sequence SEQ ID NO. 1.

### PRIORITY

This application claims priority under 35 U.S.C. §120 as a continuation-in-part of International Application number PCT/BE01/00162, filed Sep. 21, 2001, which claims priority to European application number 00870211.0, filed Sep. 22, 2000.

### FIELD OF THE INVENTION

The present invention is related to newly identified, isolated and purified members of the family of olfactory ligand and the family of pheromone ligand G-protein-coupled receptors (preferably human) as well as to the various uses that can be made of said receptors.

The invention is also related to the polynucleotides sequences encoding the pheromone receptors of the invention.

The invention is further related to methods using receptor polypeptides and polynucleotides applicable to diagnostic and treatment in receptor-mediated disorders.

The invention is further related to ligand-screening methods using the receptor polypeptides and polynucleotides, to identify agonists and antagonists useful to improve flavors or perfumes. Ligands of the pheromone receptors of the invention may also be used in the prevention and/or treatment of various disorders.

The invention further encompasses agonists and antagonists based on the said olfactory and pheromones receptor polypeptides and polynucleotides as well as biosensors comprising said receptor polypeptides.

The invention is further related to procedures for producing the receptor polypeptides and polynucleotides according to the invention, preferably by genetic recombinant methods.

### BACKGROUND OF THE INVENTION

G-protein coupled receptors (GPCRs) are proteins responsible for transducing a signal within a cell. GPCRs have usually seven transmembrane domains. Upon binding of a ligand to an extra-cellular portion or fragment of a GPCR, a signal is transduced within the cell that results in a change in a biological or physiological property or behaviour of the cell. GPCRs, along with G-proteins and effectors (intracellular enzymes and membrane channels modulated by G-proteins), are the components of a modular signaling system that connects the state of intra-cellular second messengers to extra-cellular inputs.

GPCR genes and gene products are potential causative agents of disease and these receptors seem to be of critical importance to both the central nervous system and peripheral physiological processes.

The GPCR protein superfamily is represented in five families: Family I, receptors typified by rhodopsin and the beta2-adrenergic receptor and currently represented by over 200 unique members; Family II, the parathyroid hormone/calcitonin/secretin receptor family; Family III, the metabotropic glutamate receptor family, Family IV, the CAMP receptor family, important in the chemotaxis and development of D. discoideum; and Family V, the fungal mating pheromone receptor such as STE2.

G proteins represent a family of heterotrimeric proteins composed of α, β and γ subunits, that bind guanine nucleotides. These proteins are usually linked to cell surface receptors (receptors containing seven transmembrane domains).

Following ligand binding to the GPCR, a conformational change is transmitted to the G protein, which caused the α-subunit to exchange a bound GDP molecule for a GTP molecule and to dissociate from the βγ-subunits.

The GTP-bound form of the α, β and γ-subunits typically functions as an effector-modulating moiety, leading to the production of second messengers, such as cAMP (e.g. by activation of adenyl cyclase), diacylglycerol or inositol phosphates.

Greater than 20 different types of α-subunits are known in humans. These subunits associate with a small pool of β and γ subunits. Examples of mammalian G proteins include Gi, Go, Gq, Gs and Gt. G proteins are described extensively in Lodish et al., Molecular Cell Biology, (Scientific American Books Inc., New York, N.Y., 1995), the contents of which are incorporated herein by reference.

Known and unknown GPCRs constitute now major targets for drug action and development.

More than 300 GPCRs have been cloned thus far and it is generally assumed that it exists well over 1000 such receptors. Mechanistically, approximately 50-60% of all clinically relevant drugs act by modulating the functions of various GPCRs (Cudermann et al., J. Mol. Med., Vol. 73, pages 51-63, 1995).

### SUMMARY OF THE INVENTION

The present invention is related to newly isolated and purified identified members of olfactory and pheromone GPCRs (SEQ ID NO. 1 to SEQ. ID NO. 254) as well as to polynucleotide sequences, including recombinant sequences, encoding said receptors, described hereafter.

The present invention is also related to nucleotide and/or amino acid sequences homologous to the sequences corresponding to the receptors described hereafter.

Homologous sequences mean sequences which present a high sequence identity (which present an identity higher than 75%, 80%, 85%, 90% or 95%) with the complete sequence described hereafter.

Homologous sequences of a sequence according to the invention may include an amino acid or nucleotide sequence encoding a similar receptor which exists in other animal species (rat, mouse, cat, dog, etc.) or in specific human population groups, but which are involved in the same biochemical pathway(s).

Such homologous sequences may comprise additions, deletions or substitutions of one or more amino acids or nucleotides, which do not substantially alter the functional characteristics of the receptor according to the invention. For example, homologs of GPCRs of the present invention will have at least 90% of the activity of wild-type full length human olfactory and/or pheromone GPCRs of the invention.

Such homologous sequences can also be nucleotide sequences of more than 400, 600, 800 or 1000 nucleotides which are able to hybridize to the complete nucleic acid sequences of SEQ ID Nos.: 1-254 under stringent hybridization conditions (such as the ones described by SAMBROOK et al., Molecular Cloning, Laboratory Manuel, Cold Spring, Harbor Laboratory press, New York). An example of “stringent hybridization conditions” is as follows: hybridize in 50% formamide, 5×SSC, 50 mM sodium phosphate (pH 6.8), 0.1% sodium pyrophosphate, 5×Denhardt's solution, 50 μg/ml sonicated salmon sperm DNA, 0.1% SDS and 10% dextran sulfate at 42° C.; and wash at 42° C. (or higher, e.g., up to two degrees C. below the Tₘ of the perfect complement of the probe sequence) in 0.2×SSC and 0.1% SDS.

Another aspect of the present invention is related to a specific active portion of said sequences or a libraries of active portions of these sequences. Active portions could be partial or deleted receptors which comprise modifications (e.g., point mutations) or deletions upon the complete nucleotide or amino acid sequences and which still maintain the active site(s) necessary for the binding of a specific ligand able to interact with said receptors.

As used herein, an “active portion” refers to a portion of a sequence that is of sufficient size to exhibit normal or near normal pharmacology (e.g., receptor activity, the response to an activator or inhibitor, or ligand binding are at least 90% of the level of activity, response, or binding exhibited by a wild type receptor). “A portion” as it refers to a sequence encoding a receptor, refers to less than 100% of the sequence (i.e., 99, 90, 80, 70, 60, 50% etc . . . ). The active portion could be a receptor which comprises a partial deletion of the complete nucleotide or amino acid sequence and which still maintains the active site(s) and protein domain(s) necessary for the binding of and interaction with a specific ligand.

Homologous sequences of the sequences according to the invention may comprise similar receptors which exist in other animal (vertebrates, preferably mammalian) or specific human populations, but which are involved in the same biochemical pathway.

Such homologous sequences may comprise addition, deletion or substitution of one or more amino acids or nucleotides, which does not substantially alter the functional characteristics of the receptor(s) according to the invention in order to form preferably a hybrid polypeptide with another transmembrane protein, suitable for rapid ligand screening.

Thus, the invention encompasses also a receptor and corresponding nucleotide sequence which may be derived from olfactory neurons or olfactory epithelium, having exactly the same amino acid or nucleotide sequences as shown in the enclosed sequence listing (SEQ ID Nos 1-254).

The invention is preferably related to human receptors characterized by the complete nucleotide and amino acid sequences described hereafter, agonist and antagonist compounds or inhibitors, and antibodies or specific hypervariable portions thereof that bind specifically to the receptors (i.e. that have at least a 10 fold greater affinity for said receptors than any other naturally occurring antibody). The invention relates, with particularity to antibodies made by a process comprising the injection of a pharmaceutically acceptable preparation of an amino acid sequence of a receptor according to the invention (or an active portion thereof) into a animal capable of producing antibodies directed against said receptor. Alternatively, antibody polypeptide molecules may be generated by injecting an animal with a DNA expression vector comprising a nucleic acid sequence encoding a receptor polypeptide of the invention, and appropriate control sequences, such that upon expression of the receptor, an immune response is raised in the host animal, thereby generating antibody polypeptide molecules to the receptor polypeptide.

For instance, a monoclonal antibody to the receptor according to the invention may be obtained by injecting an expression plasmid comprising the DNA encoding said receptor into a non-human mammal and than fusing mouse spleen cells with myeloma cells.

The present invention is also related to a polynucleotide molecule encoding an olfactory and/or pheromone receptor polypeptide according to the invention, possibly linked to other expression sequences and incorporated into a vector (e.g., plasmid, virus such as an adenovirus, liposomes, cationic vesicles). The invention also relates to host cells transformed by such a vector.

The present invention is also related to the recombinant, preferably human, receptor according to the invention, produced by such host cells according to the methods well known by the person skilled in the art.

The present invention is also related to a transgenic non human mammal comprising a partial or total deletion of the genetic sequences encoding the receptor according to the invention, preferably a non human mammal comprising an homologous recombination “knock-out” of the nucleotide sequences (polynucleotides) according to the invention or a transgenic non human mammal overexpressing above natural level said nucleotide sequences (polynucleotides).

A transgenic non human mammal can be obtained by methods well known by the person skilled in the art, for instance by the one described in the document WO98/20112 using classical techniques based upon the transfection of embryonic stem cells, preferably according to the method described by Carmeliet et al., Nature, Vol. 380, p. 435-439, 1996.

Preferably, a transgenic non-human mammal overexpressing the polynucleotides according to the invention or portions thereof comprises the polynucleotides or active portions thereof incorporated in a DNA construct with an inducible promoter allowing its overexpression and possibly tissues and other specific regulatory elements.

As used herein. “overexpress” refers to a level that is at least 2-fold, preferably 5-fold, more preferably 10-fold and most preferably 100-fold or more (i.e., 150-fold, 200-fold, 250-fold, 500-fold, 1000-fold, 10,000-fold etc..) as compared to the level of expression of the endogenous receptor in its normal native context. A transgenic non-human mammal according to the invention will express the transgene in at least one tissue or cell type but can express a transgene (e.g., a transgene encoding a pheromone receptor of the present invention) in all tissues and cells.

Another aspect of the present invention is related to a method for screening for compounds which bind to the receptor polypeptide molecules according to the invention, said method comprising providing a cell containing a nucleic acid molecule encoding an olfactory/pheromone receptor of the invention or a portion thereof and expressing the receptor or portion thereof; exposing said cell to a candidate compound; and detecting the presence of a compound bound to said receptor or portion thereof, thereby determining whether said compound binds to said receptor or portion thereof.

In one embodiment, the method further comprises the step of preparing a cell extract from the cell transfected with said nucleic acid molecule, isolating a membrane fraction of the cell extract, and contacting the candidate compound with the membrane fraction under conditions permitting binding of the compound to said fraction.

In a further embodiment, the invention comprises isolating and/or recovering the candidate compound which binds to the receptor.

The present invention provides further, a method for identifying a compound as a ligand, agonist, or antagonist of a GPCR according to the invention comprising; providing a cell containing a nucleic acid molecule encoding an olfactory/pheromone receptor of the invention or a portion thereof and expressing the receptor or portion thereof; exposing said cell to a candidate compound; and detecting a signaling activity of the receptor or portion thereof, wherein if a signaling activity is detected, then the candidate compound is identified as a ligand, agonist, or antagonist of the GPCR.

In a further embodiment, the invention comprises isolating and/or recovering the candidate compound which is a ligand of a receptor of the invention and which is identified by the above method.

As used herein, an “antagonist” is a ligand which competitively binds to a receptor at the same site as an agonist, but does not activate an intracellular response initiated by an active form of the receptor. An antagonist thereby inhibits the intracellular response induced by an agonist by at least 10%, preferably 15-25%, more preferably 25-50% and most preferably, 50-100%, as compared to the intracellular response in the presence of an agonist and in the absence of an antagonist.

As used herein, an “agonist” refers to a ligand that activates an intracellular response when it binds to a receptor at concentrations equal to or lower than angiopeptin concentrations which induce an intracellular response. An agonist according to the invention can increase the intracellular response mediated by a receptor by at least 2-fold, preferably 5-fold, more preferably 10-fold and most preferably 100-fold or more (i.e., 150-fold, 200-fold, 250-fold, 500-fold, 1000-fold, 10,000-fold etc . . . ), as compared to the intracellular response in the absence of agonist. An agonist according to the invention may decrease internalization of a cell surface receptor such that the cell surface expression of a receptor is increased by at least 2-fold, preferably 5-fold, more preferably 10-fold and most preferably, 100-fold or more (i.e., 150-fold, 200-fold, 250-fold, 500-fold, 1000-fold, 10,000-fold etc . . . ), as compared to the number of cell surface receptors present on the surface of a cell in the absence of an agonist. In another embodiment of the invention, an agonist stabilizes a cell surface receptor and increases the cell surface expression of a receptor by at least 2-fold, preferably 5-fold, more preferably 10-fold and most preferably, 100-fold or more (i.e., 200-fold, 250-fold, 500-fold, 1000-fold, 10,000-fold etc . . . ), as compared to the number of cell surface receptors present on the surface of a cell in the absence of agonist.

An “inhibitor” compound according to the invention is a molecule directed against the receptor or against the natural ligand for the receptor that decreases the binding of the ligand to the receptor by at least 10%, preferably 15-25%, more preferably 25-50% and most preferably, 50-100%. An “inhibitor” compound of the invention can decrease the intracellular response induced by an agonist by at least 10%, preferably 15-25%, more preferably 25-50% and most preferably, 50-100%. An “inhibitor” also refers to a nucleotide sequence encoding an inhibitor compound of the invention.

As used herein, the term “receptor signaling activity” refers to the initiation or propagation of signaling by an olfactory/pheromone receptor polypeptide of the invention. Receptor signaling activity is monitored by measuring a detectable step in a signaling cascade by assaying one or more of the following: stimulation of GDP for GTP exchange on a G protein; alteration of adenylate cyclase activity; protein kinase C modulation; phosphatidylinositol breakdown (generating second messengers diacylglycerol, and inositol triphosphate); intracellular calcium flux; activation of MAP kinases; modulation of tyrosine kinases; or modulation of gene or reporter gene activity. A detectable step in a signaling cascade is considered initiated or mediated if the measurable activity is altered by 10% or more above or below baseline. The measurable activity can be measured directly, as in, for example, measurement of cAMP or diacylglycerol levels. Alternatively, the measurable activity can be measured indirectly, as in, for example, a reporter gene assay.

As used herein, “ligand” refers to a moiety that is capable of associating or binding to a receptor. According to the method of the invention, a ligand and a receptor have a binding constant that is sufficiently strong to allow detection of binding by an assay method that is appropriate for detection of a ligand binding to a receptor (e.g. a second messenger assay to detect an increase or decrease in the production of a second messenger in response to ligand binding to the receptor, a binding assay to measure protein-ligand binding or an immunoassay to measure antibody-antigen interactions). A ligand according to the invention includes the natural ligand that binds a receptor, or a ligand may be any nucleotide, antibody, antigen, enzyme, peptide, polypeptide or nucleic acid capable of binding to the receptor. According to the method of the invention, a ligand and receptor specifically bind to each other (e.g. via covalent or hydrogen bonding or via an interaction between, for example, a protein and a ligand, an antibody and an antigen or protein subunits). Specifically, a ligand according to the invention includes an odorant, flavor, or pheromone.

As used herein, the terms “change”, “difference”, “decrease”, or “increase” as applied to e.g., binding or signaling activity or amount of a substance refer to an at least 10% increase or decrease in binding, signaling activity, or for example, level of mRNA, polypeptide or ligand relative to a standard in a given assay.

As used herein, the “second messenger assay” preferably comprises the measurement of guanine nucleotide binding or exchange, adenylate cyclase, intra-cellular cAMP, intracellular inositol phosphate, intra-cellular diacylglycerol concentration, intracellular calcium concentration (e.g., via an aequorin based assay), arachinoid acid concentration, MAP kinase(s) or tyrosine kinase(s), protein kinase C activity, or reporter gene expression according to methods known in the art and defined herein.

As used herein, the term “second messenger” refers to a molecule, generated or caused to vary in concentration by the activation of a G-Protein Coupled Receptor, that participates in the transduction of a signal from that GPCR. Non-limiting examples of second messengers include cAMP, diacylglycerol, inositol triphosphate, arachidonic acid release, inositol triphosphates and intracellular calcium. The term “change in the level of a second messenger” refers to an increase or decrease of at least 10% in the detected level of a given second messenger relative to the amount detected in an assay performed in the absence of a candidate modulator.

As used herein, the term “binding” refers to the physical association of a ligand (e.g., angiopeptin, or an antibody) with a receptor (e.g., pheromone GPCR). As the term is used herein, binding is “specific” if it occurs with an EC₅₀ or a K_{d} of 1 mM less, generally in the range of 1 mM to 10 nM. For example, binding is specific if the EC₅₀ or K_{d} is 1 mM, 500 μM, 100 μM, 10 μM, 9.5 μM, 9 μM, 8.5 μM, 8 μM, 7.5 μM, 7 μM, 6.5 [μM, 6 μM, 5.5 μM, 5 μM, 4.5 μM, 4 μM, 3.5 μM, 3 μM, 2.5 μM, 2 μM, 1.5 μM, 1 μM, 750 nM, 500 nM, 250 nM, 100 nM, or 50 nM or less.

The present invention is related to molecules or compounds, preferably pheromones, odorants, or flavors, including possible toxic molecules identified by the screening methods of the invention and to their pharmaceutical, cosmetic and industrial (e.g., production of detergents, soap, shampoo, fragrances, odorant fingerprints, appetite suppressant compounds, etc.) use.

Such molecules or compounds may be used also for modifying taste and/or physiological reactions to odorant, pheromones or flavors in a mammal.

The present invention is also related to a (preferably nasal) spray for controlling appetite comprising the identified compounds or molecules by the method according to the invention in a suitable carrier.

Another application of such receptor is the trapping of odor by using the receptor, the cell or membrane according to the invention wherein the desired odor ligand is absorbed by the binding of the odorant ligand to the odorant receptor.

The present invention is further related to an odor trap, using said method for trapping odor.

Preferably, the tested odorants or pheromones upon the receptor according to the invention are advantageously selected from different body secretions such as sweat, salivary, urine, vaginal secretions, sperm, etc. The tested odorants or pheromones upon the receptor are also advantageously selected from the group of 16-androstene family, such as the 5α-androst-16-en-3α-ol, the 5α-androst-16-en-3-one, androstadienone, a human pheromone described previously (Grosser et al., Psychoneuroendocrinology vol. 25, pages 289-299, 2000) and other androstenol derivatives; the group consisting of estrene family, such as 1,3,5(10),16-estratetraen-3-ol and other estradiol derivatives described in PCT/US92/00219, PCT/US92/00220 and EP0562843; the group consisting of progestin family such as the human pheromone pregna-4,20-diene-3,6-dione (Monti-Bloch et al., J. Steroid Biochem. Mol. Biol. Vol. 65, pages 237-242) and other progesterone derivatives, the group consisting of small fatty acids such as acetic acid, propionic acid, butyric acid, isovaleric acid and isocaproïc acid that compose the putative human vaginal pheromone copulin and such as the trans-3-methyl-2-hexanoic acid found in human sweat, cyclic organic compounds homologs to known animal pheromone such as dehydro-exo-brevicomin and such as nepetalactone, and human homologs of the murin protein aphrodisin.

Other examples of such compounds are molecules present in vapour emanating from narcotics, like cocaine, marihuana, heroin, hashish, angel dust, gasoline, natural gas, alcohol, decayed human flesh, explosives, plastic explosives, fire arms, gun powder, toxic fumes, noxious fumes or dangerous fumes, etc.

Molecules or compounds which bind to and/or activate the receptors of the invention could be used also for promoting or suppressing chemical communication. Compounds which bind to and/or activate the receptor polypeptides of the invention are also useful as emitted stimuli able to modify the probability of response of a first organism or a part of a first organism when released by a second organism or an exogenous source.

The molecules or compounds of the invention could be used in the treatment or prevention of various disorders affecting, for instance, cell migration, cell death, cell growth, psychotic and neurological disorders, including anxiety, schizophrenia, maniac depression, depression or mound modification, etc.

As used herein, a “therapeutic amount” of a compound is an amount that, when contacted with a receptor of the present invention, increases or decreases ligand binding to said receptor or activation of said receptor by at least 10%, preferably 20%, 30%, 50% and up to 100%.

The receptors and/or ligands of the invention could be used also for improving contraceptive medication, treatment promoting axonal growth, neural connection and nerves regeneration, in modulating male and female endocrine functions, or may effect the menstrual cycle. Indeed, it is known that several of said receptors can be present at the surface of spermatozoids and can then find contraceptive application or could be used in the of sterility/fertility. these receptors are also present in various neurolfactory neurons and can improve their connection, especially upon the olfactory bulb or upon other tissue comprising said olfactory receptors.

Receptors and/or ligands of the invention could be also used for the treatment or the prevention of various animal or human behaviours, such as stimulation and/or suppression of appetite, stimulation and/or suppression of motivation and sexual attraction, stimulation and/or suppression of aggressivity, stimulation and/or suppression of alarm and defense behaviours, stimulation and/or suppression of territory and trail-marking, stimulation and/or suppression of social regulation and recognition, stimulation and/or suppression of mother-child recognition, etc.

The screening method according to the invention could be performed by well known methods to the person skilled in the art, preferably high-throughput screening, diagnostic and dosage devices based upon the method described in the International patent application WO00/02045 performed upon various solid supports such as micro-titer plates or biochips according to known techniques by the person skilled in the art.

Methods for screening for the binding of a candidate compound to a receptor molecule of the invention may be performed by several techniques which are known to those of skill in the art including, but not limited to surface plasmon resonance (Salamon et al., 1996, Biophys J. 71: 283-294; Salamon et al., 2001, Biophys. J. 80: 1557-1567; Salamon et al., 1999, Trends Biochem. Sci. 24: 213-219; Sarrio et al., 2000, Mol. Cell. Biol. 20: 5164-5174), or FRET.

Methods for screening for receptor signaling activity are also known to those of skill in the art and include, but are not limited to GTPase/GTP binding assays (Traynor and Nahorski, 1995, Mol. Pharmacol. 47: 848-854), aequorin assays which measure the response of mitochondrial apoaequorin to intracellular calcium release induced by the activation of GPCRs (Stables et al., 1997, Anal. Biochem. 252:115-126; Detheux et al., 2000, J. Exp. Med., 192 1501-1508), adenylate cyclase assays(Kenimer & Nirenberg, 1981, Mol. Pharmacol. 20: 585-591), cAMP assay (Horton & Baxendale, 1995, Methods Mol. Biol. 41: 91-105), assays for PLC breakdown and DAG/ inositol triphosphate (IP₃) production )(Phospholipid Signaling Protocols, edited by Ian M. Bird. Totowa, N.J., Humana Press, 1998; Rudolph et al., 1999, J. Biol. Chem. 274: 11824-11831), PKC activation assay (Kikkawa et al., 1982, J. Biol. Chem. 257: 13341), kinase assays, transcriptional reporter assays, or other functional assays include, for example, microphysiometer or biosensor assays (Hafner, 2000, Biosens. Bioelectron. 15: 149-158).

The present invention is also related to the molecules characterized and possibly recovered by said method, including the pharmaceutical composition comprising a sufficient amount of said molecules and a pharmaceutically acceptable carrier or diluent for the preparation of a medicament in the prevention and/or the treatment of specific diseases.

A last aspect of the present invention is related to a biosensor or any technical device comprising the receptors according to the invention for the detection of the specific above-mentioned compounds or molecules.

1. Isolated pheromone G-protein coupled receptor comprising the sequence of SEQ ID NO: 1.

2. Isolated G-protein coupled receptor having the amino acid sequence SEQ ID NO: 1 or an active portion thereof.

3. An isolated polynucleotide encoding the G-protein coupled receptor of claim 1 or 2.

4. A vector comprising the polynucleotide according to the claim 3.

5. A cell comprising the vector according to the claim 4.

6. Non-human mammal comprising a deletion of the polynucleotide according to the claim 3.

7. The non-human mammal of claim 6 wherein said animal is preferably a non-human mammal.

8. A transgenic non-human mammal which overexpresses the polynucleotide of claim 3.

9. A method for screening for compounds which bind to the receptor polypeptide molecules according to the invention, said method comprising
providing a cell containing a nucleic acid molecule encoding an olfactory/pheromone receptor of the invention or a portion thereof and expressing the receptor or portion thereof;
exposing said cell to a candidate compound; and
detecting the presence of a compound bound to said receptor or portion thereof, thereby determining whether said compound binds to said receptor or portion thereof.

10. The method of claim 9, further comprising the steps of preparing a cell extract from the cell transfected with said nucleic acid molecule; isolating a membrane fraction of the cell extract; and contacting the candidate compound with the membrane fraction under conditions permitting binding of the compound to said fraction.

11. The method of claim 9, further comprising the step of isolating and/or recovering the candidate compound which binds to the receptor.

12. A method for identifying a compound as a ligand, agonist, or antagonist of a GPCR according to the invention comprising;
providing a cell containing a nucleic acid molecule encoding an olfactory/pheromone receptor of the invention or a portion thereof and expressing the receptor or portion thereof;
exposing said cell to a candidate compound; and
detecting a signaling activity of the receptor or portion thereof, wherein if a signaling activity is detected, then the candidate compound is identified as a ligand, agonist, or antagonist of the GPCR.

13. The method of claim 12, wherein following detection of a signaling activity, said candidate compound is isolated and/or recovered.

14. Pharmaceutical composition comprising an adequate pharmaceutical carrier and a therapeutic amount of the receptor according to claim 1 or 2, the vector according to the claim 5, or the cell according to the claim 6.
